(19) United States
(12) Patent Application Publication
(10) Pub. No.: US 2008/0138842 A1
(43) Pub. Date: Jun. 12, 2008

(54) Indirect lateral flow sandwich assay

(76) Inventors: Hans Boehringer, San Diego, CA (US); Mark Daquipa, San Diego, CA (US); Fon-Chiu Mia Chen, Ramona, CA (US); Hsin Ming Yang, San Diego, CA (US); Thomas L. Pisani, Winchester, MA (US); Sumitra Nag, Lexington, MA (US); Jay Salhaney, Holliston, MA (US); Marcella B. Holdrige, Watertown, MA (US); Erika Johnston, Lexington, MA (US); Jeremy Schonhorn, Hopkinton, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/983,671

(22) Filed: Nov. 9, 2007

Related U.S. Application Data
(60) Provisional application No. 60/874,302, filed on Dec. 11, 2006.

(51) Int. Cl. C12M 1/34; G01N 33/00

(52) U.S. Cl. 435/7.94; 435/287.2

(57) ABSTRACT

Disclosed herein are indirect lateral flow sandwich assays, in which the target analyte binds an analyte-specific reagent comprising a first member of a conjugate pair, forming a complex which contacts and binds a colored particulate label comprising a complementary member of said conjugate pair, forming a second complex. Capture of this analyte-comprising, second complex by an immobilized analyte specific capture reagent results in the formation of an immobilized labeled sandwich complex that can be detected.

### CROSS-REFERENCE TO RELATED APPLICATION

This application claims the benefit of provisional patent application Ser. No. 60/874,302, filed Dec. 11, 2006 under 35 U.S.C. §119(e), which is hereby incorporated by reference in its entirety.

### BACKGROUND OF THE INVENTION

The assays, devices and methods described herein relate to the detection of an analyte in a liquid, including a bodily fluid.

In a lateral flow device, the sample, which comprises an analyte of interest, is permitted to flow laterally from the point of its application through one or more regions of one or more membrane surfaces to a detection zone. The presence of an analyte in the applied sample can be detected by a variety of protocols, including direct visualization of visible moieties associated with the captured analyte. Deutsch et al. describe a chromatographic test strip device in U.S. Pat. Nos. 4,094,647, 4,235,601 and 4,361,537. The device comprises a material capable of transporting a solution by capillary action, i.e., wicking. Different areas or zones in the strip contain the reagents needed to produce a detectable signal as the analyte is transported to or through such zones.

In addition, European Patent Publication No. 0 323 605 B1 discloses an assay device which uses chromatographic material in a sandwich assay to detect an analyte.

U.S. Pat. No. 6,368,876 discloses an immunochromatographic assay device that comprises a separated sample receiving region which is made of a porous material. The porous material conducts lateral flow of the liquid sample. The sample receiving region is in contact with a separate analyte detection region. Lateral flow of the liquid sample will continue from the sample receiving region to the analyte detection region. The analyte detection region contains a porous material which permits lateral flow of the liquid sample. The analyte detection region contains mobile labeling reagents located at a discrete situs. It also contains an immobile capture reagent at a discrete situs. In addition, it also contains a control reagent at a discrete control situs. In the disclosure of U.S. Pat. No. 6,368,876, the analyte detection region is also in lateral flow contact with the end flow region. The end flow region contains a porous material capable of absorbing excess liquid sample and which facilitates lateral flow of the liquid sample.

### SUMMARY OF THE INVENTION

The assays, devices and methods described herein relate to the detection of one or more analytes in an liquid solution using at least one conjugate which comprises colored particles and which are not specific for the analyte. The use of particulate labels provides a high degree of sensitivity to the assays, and avoids the need for secondary reagents for analyte detection. The assays, devices and methods described herein provide a means to achieve a highly sensitive, rapid and reliable determination of the presence of an analyte in a liquid solution.

Described herein are immunochromatographic assay devices and assays for detecting the presence or absence of an analyte in a liquid sample, preferably an aqueous solution, using a lateral flow assay. In one embodiment, the lateral flow assay comprises the use of a device which contains a test strip on which are located mobilizable colored particles which do not specifically bind the analyte(s), in a separate location from mobilizable analyte-specific antibody. Also described herein are methods of making and using these devices.

One embodiment disclosed herein is an indirect sandwich lateral flow assay for detecting the presence of an analyte in a liquid, in which an analyte of interest specifically binds an analyte-specific reagent, preferably an analyte-specific antibody, where the reagent comprises a first member of a conjugate pair, forming a first complex. This first complex contacts and binds a colored particulate label comprising a second member of the conjugate pair, i.e. a complementary member, forming a second complex. This second complex comprises the first complex bound to the colored particle label through the first and second conjugate members. Capture of this second complex by a capture reagent which specifically binds analyte and which is immobilized on the assay strip/membrane results in the formation of an immobilized, detectable sandwich complex comprising the analyte of interest.

Described herein is a device for detecting an immunoreactive analyte present in an aqueous solution. In the dry, unused state, the device does not comprise a particle-labeled reagent capable of specifically binding analyte. However, upon addition of liquid sample, a particle labeled conjugate capable of specifically reacting with antigen is formed. This particle labeled conjugate can migrate and, provided analyte is present, be captured in a downstream detection zone by an immobilized analyte specific reagent.

In one embodiment, the device comprises a first pad (conjugate pad) and a detection zone on a separate membrane surface or strip, the first pad (conjugate pad) and the detection zone being positioned to permit capillary flow of an aqueous solution from the first pad (conjugate pad) to the detection zone on the separate pad. The first pad (conjugate pad) comprises a porous structure through which an aqueous solution is capable of flowing by capillary action. The aqueous solution may have dissolved in it one or more of the following species at various time points during the assay: one or more analytes of interest, one or more antibody-analyte complexes, and/or one or more complexes comprising an antibody.

The first pad (conjugate pad) has a first zone and a second zone, which are preferably adjacent or slightly separated from each other. The first zone contains a dry, reversibly immobilized reagent specific for the analyte, preferably an antibody specific for the analyte, the reagent or antibody further comprising a first member of a conjugate pair. The second zone of the conjugate pad contains a dry, reversibly immobilized, colored particulate label, which also contains a complementary member of the conjugate pair. Alternatively, the first and second zones can be reversed, e.g., the first zone containing a dry, reversibly immobilized, colored particulate label, which also contains a complementary member of the conjugate pair, and the second zone containing a dry, reversibly immobilized reagent specific for the analyte, preferably an antibody specific for the analyte, the reagent or antibody further comprising a first member of a conjugate pair.

Located downstream from the first pad (conjugate pad) with respect to capillary flow in such devices, is a detection zone on a second pad, which has a capture line containing an irreversibly immobilized capture reagent capable of specifically binding to the analyte, preferably a capture antibody capable of specifically binding to the analyte.

Also described herein are methods of detecting an analyte in an aqueous solution through use of devices described herein. Upon applying aqueous sample comprising or suspected to comprise an analyte of interest to the first pad (conjugate pad) of the device, dry, reversibly immobilized analyte specific reagent is reconstituted and mobilized, forming a first complex with analyte, if present, the complex containing the analyte-specific reagent or antibody, bound to the analyte. This first complex, together with mobilized, unbound antibody, is capable of moving by capillary action to the second zone of the first pad (conjugate pad), where the first complex binds to the colored particulate label through the interaction of the first and second members of the conjugate pair, resulting in the formation of a second, three member complex. The second complex, containing the first complex bound to the particulate label, subsequently moves by capillary action to the capture line located in the detection zone located on a separate pad or substrate.

The second complex specifically binds to the capture antibody accumulating as a fourth sandwich complex at the capture line. The formation of the sandwich complex is indicative of the presence of the analyte of interest in the aqueous sample, and can be detectable by any means suited to detection of the colored particle component, preferably by the naked eye.

Controls for the formation and sufficient migration of the particle-labeled, analyte-specific reagent can take different forms. In one embodiment, the unbound analyte-specific antibody also binds the colored particulate label in said second zone, forming a third complex. The third complex comprises the colored particulate label and analyte-specific antibody which has no analyte bound to it. This third complex also moves by capillary action to the detection zone, but, lacking analyte, it passes the capture line, and is captured at the control line of the detection zone by a reagent that binds the antibody of the complex.

Alternatively, the conjugate pad can contain a dry, reversibly immobilized non-analyte reagent. The non-analyte reagent can be any substance, protein, enzyme or antibody on a particulate label, which reagent does not react with the analyte-specific reagent system. Typical non-analyte reagents are bovine serum albumin, goat serum albumin, mouse serum albumin, etc. The non-analyte reagent is dried onto the conjugate pad along with the analyte-specific reagent. In preferred embodiments, the particulate labels for non-analyte reagent and analyte-specific reagent are different colors, e.g, blue and red. The reagents are homogeneous at this stage. Upon applying aqueous sample, the analyte-specific and non-analyte reagents are reconstituted and mobilized. Once the mixture migrates to the detection zone, the non-analyte reagent binds to a second capture line (control line) where the complimentary binding partner, e.g., an antibody or other specific binding partner for the non analyte reagent, is irreversibly immobilized.

In one aspect, then, the analyte-specific reagent of the first zone of the first pad (conjugate pad) is an antibody, and the control line comprises a reagent which specifically binds antibodies. In one aspect, the control line is located downstream of the capture line with respect to the capillary flow of the aqueous solution. In another aspect, the reagent at the control line comprises a final capture antibody, wherein the final capture antibody specifically binds to antibody molecules. In another aspect, the final capture antibody binds antibody regardless of its specificity.

In a further aspect, then, a non-analyte, particle-labeled reagent is included on the conjugate pad and is captured in a control line bearing immobilized reagent specific for the non-analyte reagent.

In aspects where there are multiple analyte-specific reagents, each specific for one of a multiplicity of different analyte of interest, there can be multiple control lines, each line containing one or more reagents specific for at least one of the multiple reagents that serves as a control for proper reconstitution and migration of particle-labeled reagent.

In another, less preferred embodiment, this device can further comprise a sample application pad, the sample pad facilitating sample application and having a porous structure through which an aqueous solution comprising one or more analytes of interest is capable of flowing by capillary action, and positioned so as to permit an aqueous solution to flow to the first pad (conjugate pad). Where employed, the sample application pad substantially lacks analyte-specific or particle-labeled reagents.

In one embodiment of this device, the first zone of the first pad (conjugate pad) is positioned upstream of the second zone with respect to the capillary flow of the aqueous solution. In another aspect, the first zone of the first pad (conjugate pad) abuts the second zone of the first pad (conjugate pad).

While the device may be enclosed in a hollow casing or housing, in one embodiment, the assay device it is not enclosed in a hollow casing or housing.

In one embodiment of this device, the first member of the first conjugate pair is biotin and the second member of the conjugate pair is selected from the group consisting of streptavidin, neutravidin, avidin, and anti-biotin antibodies. In one aspect, when the first member is biotin, the second member of the conjugate pair is not an antibody. In another aspect, when the first member is biotin, the second member of the conjugate pair is not an antibody specific for biotin.

In one embodiment of this device, the colored particle is a latex particle or a metal sol, e.g., a colloidal gold particle, or alternatively, a carbon sol.

Also described herein is a method of detecting an analyte in an aqueous solution, the method including the step of applying an aqueous sample solution to a device as described herein. Application of the sample to the device involves and results in the following series of events:

A) contacting a sample solution with an analyte-specific antibody, reversibly immobilized to a porous structure and comprising a first member of a conjugate pair, under conditions that allow mobilization of the analyte-specific antibody and the formation of a first complex in which the analyte is specifically bound to the analyte-specific antibody;

B) as liquid sample carrying the first complex migrates down the structure from the point of application by capillary action, the sample subsequently contacts and mobilizes a colored particulate label reversibly immobilized to the porous structure and located distal to the analyte-specific reagent, the colored particle label comprising a complementary member of the conjugate pair, under conditions that permit the formation of a second complex in which the first complex is specifically bound to the colored particulate label via the interaction of the members of the conjugate pair;

C) upon formation of the second complex, through capillary movement the second complex subsequently migrates to a second porous structure or substrate, where it contacts an analyte specific capture antibody which is irreversibly immobilized to the structure at a position distal to the site of formation of the second complex, under conditions that allow the formation of a third complex comprising the second complex and the analyte-specific capture antibody; and

D) detecting the formation of the third complex by detecting its colored particulate label component accumulated in the detection zone by a detection means appropriate to the nature of the particulate label, wherein detection of the third complex indicates the presence of the analyte in the aqueous solution.

In a further aspect of this embodiment, in step (B), the analyte-specific antibody of part (A), which has not bound analyte also makes contact with the colored particulate label of part (B), under conditions that allow the formation of a fourth complex in which the analyte-specific antibody of part (A), which has not bound analyte, is specifically bound to the colored particulate label, e.g., according to the following steps (E) and (F):

E) Upon formation of the fourth complex, the fourth complex is contacted with a reagent which specifically binds to antibody molecules of any specificity and which is irreversibly immobilized to the porous structure at a position distal to the site of formation of the second complex and the third complex. The contact is under conditions that allow the formation of a fifth complex. This fifth complex comprises the fourth complex and the irreversibly immobilized reagent which specifically binds to antibody molecules;

F) The formation of the fifth complex is detected through its particulate label component accumulated on the porous structure by a detection means appropriate to the nature of the particulate label. The detection of the fifth complex acts as a control for the functionality of the assay, demonstrating that the analyte-specific, particle labeled complexes have formed and migrated at least as far as the control line. Functionally similar to events E and F are achieved in embodiments in which particle-labeled, non-analyte reagent is dried on the conjugate pad, becomes mobile with the addition of sample and migrates to a capture line of immobilized reagent specific for that non-analyte reagent.

In one aspect of any of the methods, assays, and devices described herein, the analyte is not an antibody. In one embodiment of the methods, devices and assays described herein, the complementary member of the conjugate pair is not an antibody specific for biotin. In another aspect, the methods, assays and devices described herein can detect multiple analytes of interest.

### DEFINITIONS

As described herein, the term “device” preferably encompasses a test strip comprising two pads that provide the functional elements necessary to detect analyte by adding only an aqueous sample. For example, the test strip comprises a first pad (conjugate pad), which contains a) a reversibly immobilized analyte-specific reagent with a first member of a conjugate pair, and b) a colored particulate label with a second, cognate member of the conjugate pair, and a second pad comprising a detection zone capable of specifically detecting the analyte. The detection zone can be capable of detecting one or a plurality of analytes. The device can less preferably comprise a sample pad to receive the sample. The device can also contain an absorbent pad downstream of the detection zone to provide a sink to facilitate continued capillary action and to absorb excess fluid.

As used herein, the term “porous material” or “porous structure” refers to a material capable of providing capillary movement or lateral flow. This would include material such as nitrocellulose, nitrocellulose blends with polyester or cellulose, untreated paper, porous paper, rayon, glass fiber, acrylonitrile copolymer or nylon or other porous materials that allow lateral flow. Porous materials useful in the devices described herein permit transit, either through the porous matrix or over the surface of the material, of particle label used in these devices.

The devices described herein include a test strip composed of a material which permits capillary flow of the sample solution along a flow path. By “capillary flow”, it is meant liquid flow in which all of the dissolved or dispersed components of the liquid are carried at substantially equal rates and with relatively unimpaired flow laterally through the membrane, as opposed to preferential retention of one or more components as would occur, e.g., in materials capable of adsorbing or imbibing one or more components.

As used herein, the term “lateral flow” refers to capillary flow through a material in a horizontal direction, but will be understood to apply to the flow of a liquid from a point of application of the liquid to another lateral position even if, for example, the device is vertical or on an incline. Lateral flow depends upon properties of the liquid/substrate interaction (surface wetting or wicking action) and does not require or involve application of outside forces, e.g., vacuum or pressure applications by the user.

As used herein, the term “analyte” refers to a drug, hormone, chemical, toxin, compound, receptor or other molecule and fragments thereof to be measured in the sample by the methods, kits and devices described herein. Analytes to be detected using the immunoassay devices and methods described herein include, but are not limited to, the following analytes: molecules, such as organic and inorganic molecules, peptides, proteins, glycoproteins, amino acids, carbohydrates, nucleic acids, lipids, toxins, small molecule, a steroid, a vitamin, an antibody, viruses, virus particles and the like, and combinations thereof. Analytes to be detected also include, but are not limited to, neurotransmitters, hormones, growth factors, antineoplastic agents, cytokines, monokines, lymphokines, nutrients, enzymes, receptors, antibacterial agents, antiviral agents and antifungal agents, and combinations thereof. The term “analyte” also refers to detectable components of structured elements such as cells, including all animal and plant cells, and microorganisms, such as fungi, viruses, bacteria including, but not limited to, all gram positive and gram negative bacteria, and protozoa. In one embodiment, the analyte is not an antibody. The analyte will have at least one epitope that an antibody or an immunologically reactive fragment thereof can recognize. An “analyte,” as the term is used herein can include any antigenic substances, haptens, a natural or synthetic chemical substance, a contaminant, a drug, including those administered for therapeutic purposes as well as those administered for illicit purposes, and metabolites and combinations thereof.

The term “sample” as used herein refers to any material, including any biological or organic material, that could contain an analyte for detection. Preferably the biological sample is in liquid form or can be changed into a liquid form. Preferably, the sample comprises a bodily fluid such as blood, urine, saliva, feces, secretions, cerebrospinal fluid or materials for swab based assays, etc.

As used herein, the term “application zone” or “sample pad” refers to an optional and less preferable porous structure designed to directly receive applied sample and deliver it to the first conjugate pad. The “sample pad,” if present, preferably does not include analyte-detecting or binding reagents. The liquid sample can then migrate, through lateral flow, from the application zone or sample pad towards the end flow region. The application zone can be in lateral flow contact with the first pad (conjugate pad). This can preferably be an overlap connection. That is, the application zone, when present, is contained in a separate pad that can overlap the first pad (conjugate pad), either partially or completely.

As used herein, the term “liquid” encompasses any fluid solution. As used herein, the term “aqueous solution” is any liquid comprising water. An aqueous solution can comprise salts, organic molecules, inorganic molecules, synthetic molecules, non-synthetic molecules, or any combination thereof. In one embodiment, an aqueous solution comprises one or more analytes of interest. If the analyte of interest is a solid, then the analyte is dissolved in an aqueous solution prior to being assayed. Thus, in one embodiment, a solid or semi-solid sample containing the analyte must be first diluted with an appropriate extracting or diluting solution in order to extract the analyte into an aqueous solution. An aqueous solution can also comprise one or more antibody-analyte complexes, and/or one or more complexes comprising an antibody. An aqueous solution can comprise nonaqueous components such as alcohols.

As used herein, the phrase “member of a conjugate pair” refers to a member of a conjugate pair, i.e. two molecules, usually two different molecules, where one of the molecules (i.e., a first member of a conjugate pair) through chemical or physical means specifically binds to the other molecule (i.e., a second member of a conjugate pair). The cognate or complementary members of a conjugate pair can include a ligand and its receptor; a receptor and a counter-receptor. In one aspect, a member of a cognate pair does not include an antibody specific for the other member of the pair.

As used herein, the term “reagent” encompasses substances which can be suspended or immobilized on a porous membrane or substrate and which contributes to a means for detecting analyte. For example, a “reagent” can permit visual detection of a labeled substance or substances—consider, for example, latex particles that have been bound indirectly to an analyte of interest. The label may alternatively be detected using instrumentation known to those skilled in the art such as a spectrophotometer or fluorescence detector. The reagents on the porous membrane or substrate may be immobilized or may be diffusible. Alternatively, a reagent may be diffusible such that when contacted with the sample, the reagents become mobile and move with the sample toward the distal end of the test strip or membrane.

As used herein, the term “antibody,” includes, but is not limited to a polypeptide substantially encoded by an immunoglobulin gene or immunoglobulin genes, an IgG antibody, an IgM antibody, or a portion thereof, or fragments thereof, which specifically bind and recognize an analyte, antigen or antibody. “Antibody” also includes, but is not limited to, a polypeptide substantially encoded by an immunoglobulin gene or immunoglobulin genes, or fragments thereof, which specifically bind and recognize the antigen-specific binding region (idiotype) of antibodies produced by a host in response to exposure to the analyte.

As used herein, the term “antibody,” encompasses polyclonal and monoclonal antibody preparations, as well as preparations including monoclonal antibodies, polyclonal antibodies, hybrid antibodies, phage displays, altered antibodies, F(ab′)₂ fragments, F(ab) fragments, Fᵥ fragments, single domain antibodies, chimeric antibodies, humanized antibodies, dual specific antibodies, bifunctional antibodies, single chain antibodies, and the like, and functional fragments and multimers thereof, which retain specificity for an analyte or antigen. For example, an antibody can include variable regions, or fragments of variable regions, and multimers thereof, which retain specificity for an analyte or antigen. See, e.g., Paul, FUNDAMENTAL IMMUNOLOGY, 3rd Ed., 1993, Raven Press, New York, for antibody structure and terminology. The antibody or portion thereof, may be derived from any mammalian or avian species, e.g., from a mouse, goat, sheep, rat, human, rabbit, chicken or cow antibody. An antibody may be produced synthetically by methods known in the art, including modification of whole antibodies or synthesis using recombinant DNA methodologies.

As used herein, the phrase “specifically binds to” refers to an antibody, reagent or binding moiety's binding of a ligand with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically a specific or selective reaction will be at least twice background signal to noise and more typically more than 10 to 100 times greater than background.

As used herein, the phrase “reversibly bound” or “mobilizable” refers to reagents, including antibodies, that are capable of mobility but which are releasably bound or impregnated to the assay strip. Reversibly bound reagents disperse with the liquid sample upon rehydration, becoming mobile. Upon mobilization by liquid sample, “reversibly bound” or “mobilizable” reagents are carried by the liquid sample in lateral flow. For example, the reversibly bound reagent may be a protein or molecule which recognizes or binds to an analyte and which is conjugated or attached to a first member of a conjugate pair. Another example of a reversibly bound reagent is a detectably labeled colored particle which is conjugated or attached to a second member of a conjugate pair.

As used herein, the phrase “irreversibly bound”, and the terms “immobile” or “immobilized” refer to reagents which are attached to a membrane, substrate or support such that lateral flow or capillary flow of the liquid sample does not alter the location of the immobile reagent in or on the support. Such attachment can, e.g., be through covalent, ionic or hydrophobic means. Those skilled in the art will be aware of methods available for attachment to immobilize various reagents.

As used herein, the term “label” includes a detectable indicator, including but not limited to labels which are soluble or particulate, metallic, organic, or inorganic, and may include spectral labels such as green fluorescent protein, fluorescent dyes (e.g., fluorescein and its derivatives, rhodamine) chemiluminescent compounds (e.g., luciferin and luminol), spectral calorimetric labels such as colloidal gold, or carbon particles, or colored glass or plastic (e.g. polystyrene, polypropylene, latex, etc.) beads. Where necessary or desirable, particle labels can be colored, e.g., by applying dye to particles.

As used herein, the term “colored particle label” includes, but is not limited to colored latex (polystyrene) particles, metallic (e.g. gold) sols, non-metallic elemental (e.g. Selenium, carbon) sols and dye sols. In one embodiment, a colored particle label is a colored particle that further comprises a member of a conjugate pair. Examples of colored particles that may be used include, but are not limited to, organic polymer latex particles, such as polystyrene latex beads, colloidal gold particles, colloidal sulphur particles, colloidal selenium particles, colloidal barium sulfate particles, colloidal iron sulfate particles, metal iodate particles, silver halide particles, silica particles, colloidal metal (hydrous) oxide particles, colloidal metal sulfide particles, carbon black particles, colloidal lead selenide particles, colloidal cadmium selenide particles, colloidal metal phosphate particles, colloidal metal ferrite particles, any of the above-mentioned colloidal particles coated with organic or inorganic layers, protein or peptide molecules, or liposomes.

The term “capture reagent” as used herein, refers to an immobilized (i.e., not reversibly immobilized) binding moiety which specifically recognizes or binds an analyte of interest. Preferably, a capture reagent is an analyte-specific antibody. The capture reagent is capable of forming a binding complex with the labeling reagent that has bound to analyte in the sample. The capture reagent is not affected by the lateral flow of the liquid sample due to its irreversible immobilization to the detection zone in a capture line. Once the analyte-specific capture reagent binds a complex comprising the analyte bound to analyte specific reagent which is bound to colored particulate label, the complex is prevented from continuing with the lateral flow of the liquid sample.

The term “final capture reagent” as used herein refers to any binding moiety which is capable of binding analyte specific reagent from the conjugate pad and which does not recognize or bind the analyte in the sample. The final capture reagent specifically binds, e.g., a mobile reagent of the first zone of the first pad (conjugate pad). Preferably, the final capture reagent is a protein or an antibody. In one aspect, the final capture reagent is capable of specifically binding an antibody regardless of the specificity of the antibody, e.g., a goat, anti-mouse antibody. The final capture reagent is immobilized, irreversibly bound to a control line of the detection zone. Once the final capture reagent binds a complex comprising the colored particle bound analyte specific reagent from the conjugate pad, the complex is prevented from continuing lateral flow with the liquid sample. The term “final capture reagent” also encompasses an irreversibly bound reagent that binds a non-analyte, particle-labeled control reagent that has been mobilized from the conjugate pad by the addition and migration of liquid sample.

As used herein, the phrase “control line of the detection zone” refers to a second line of reagent optionally present in the detection zone of the strip, the control line being located downstream of the capture line, and comprising a final capture reagent, preferably an antibody, to serve as a control. The final capture reagent is used, for example, to confirm that analyte specific reagent comprising a first member of a conjugate pair is reconstituted in the first zone of the first pad (conjugate pad), and further to confirm that the reagent migrates (with or without analyte) and successfully binds to the colored particulate label labeled reagent in the second zone of the first pad (conjugate pad), forming a complex comprising the conjugate pair bound to an analyte specific antibody which flows into the detection zone, passing the capture line to which the complex, lacking analyte, does not bind, and continues on its path to the immobilized final capture reagent. As an example, if the complex comprises an analyte specific antibody which is a mouse monoclonal antibody, the final capture reagent in the control line could be a rabbit anti-mouse antibody.

The term “detection zone” as used herein refers to the portion of a described assay device which is in lateral flow contact with the first pad (conjugate pad). The “detection zone” is on a separate pad from the first pad (conjugate pad). The contact between the first pad and the detection zone can preferably be an overlap connection. The first pad (conjugate pad) and the detection zone can be made of different material. In some embodiments the detection zone contains only the immobilized control and capture reagents.

The term “absorbent pad or “end flow region” as used herein refers to a portion of a described assay device which is in lateral flow contact with the detection zone, and is located downstream of the detection zone with respect to the direction of capillary movement of the applied liquid sample. Prior to use, this pad lacks reagents involved in detecting analyte. Applied liquid sample migrates towards the end flow region or absorbent pad. The end flow region or absorbent pad is capable of absorbing excess liquid sample. The end flow region can be simply an extension of the same porous material as the detection zone, or it can be a separate material or pad. Where it is a separate material or pad, the absorbent pad or end flow region can preferably overlap the detection zone, or it can abut the detection zone.

The term “housing” as used herein refers to a material, e.g. plastic, which can optionally cover the porous material of the device. In preferred embodiments there is no housing. A housing, if used, must allow the control and capture situses of the detection zone to be viewed. Thus, if the housing is clear, then the result can be viewed through the clear cover. If the housing is not clear, then a window, gap or hole must be used so the results can be viewed. In addition, the housing, if used, must leave the sample receiving region exposed so the sample can be applied to the receiving region.

As used herein, the term “reconstituted” refers to the rehydration of a dried reagent, such as an antibody or colored particulate label, which is reversibly or irreversibly bound to the test strip.

Other features and advantages of the invention will be apparent from the following detailed description of the invention in conjunction with the accompanying drawings and from the claims.

### BRIEF DESCRIPTION OF THE DRAWINGS

FIG. 1 describes an embodiment of a detectable complex containing analyte formed in the indirect sandwich assay described herein. Conventional sandwich complexes are shown on the left, and indirect complexes as disclosed herein are shown on the right. The complex comprises analyte which is bound to both an immobilized analyte-specific antibody and to a mobilized analyte-specific antibody conjugate, the antibody conjugate being bound through its conjugate member to a colored particle label comprising a complementary conjugate member.

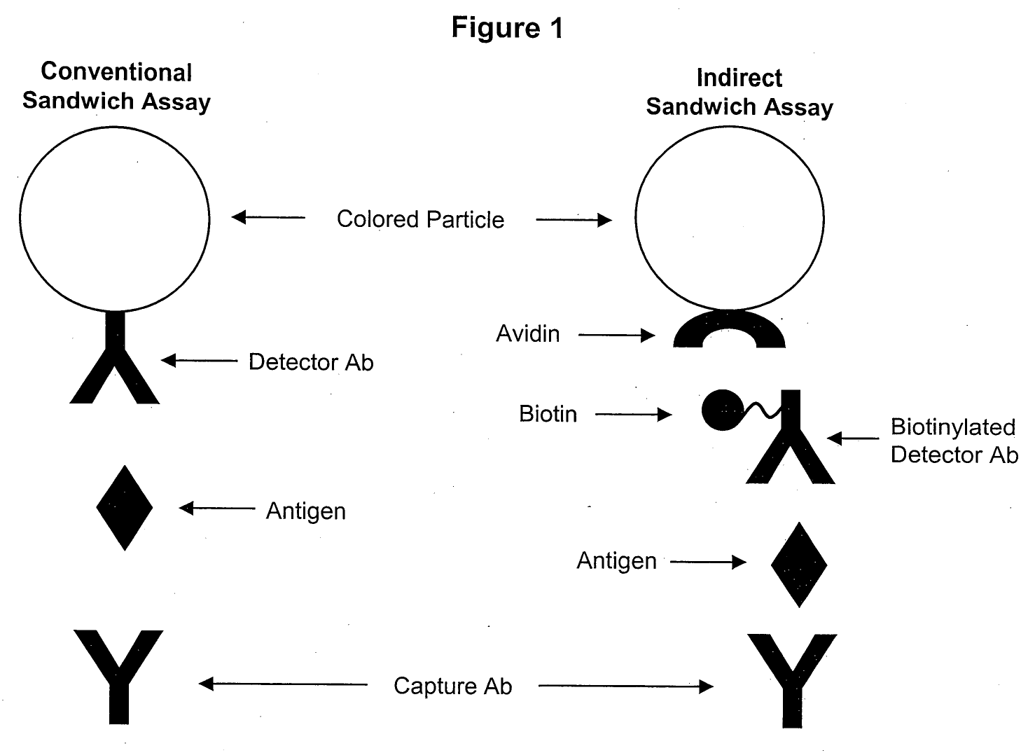

FIG. 2 describes one embodiment of an assay device described herein. The device comprises a first pad (conjugate pad) (10), a detection zone (20) and an optional absorbent pad (30). The first pad (conjugate pad) (10) comprises at least two zones: zone 1 (40) comprising a reversibly bound, mobile, analyte-specific antibody conjugated to a first member of a conjugate pair, and zone 2 (50) comprising a dry, reversibly bound, non-analyte specific, colored particle conjugated to a complementary member of the conjugate pair. The detection zone comprises a capture line (60) comprising an irreversibly bound, immobile, analyte specific reagent, and a control line (70) comprising an irreversibly bound, immobile reagent capable of specifically binding, e.g., to the analyte specific reagent of zone 1. The absorbent pad (30) comprises an absorbing material capable of acting as a wick to maintain a capillary flow of a liquid sample sequentially from zone 1 (40) of the first pad (conjugate pad) (10) through zone 2 (50), through the capture line (60), and through the control line (70).

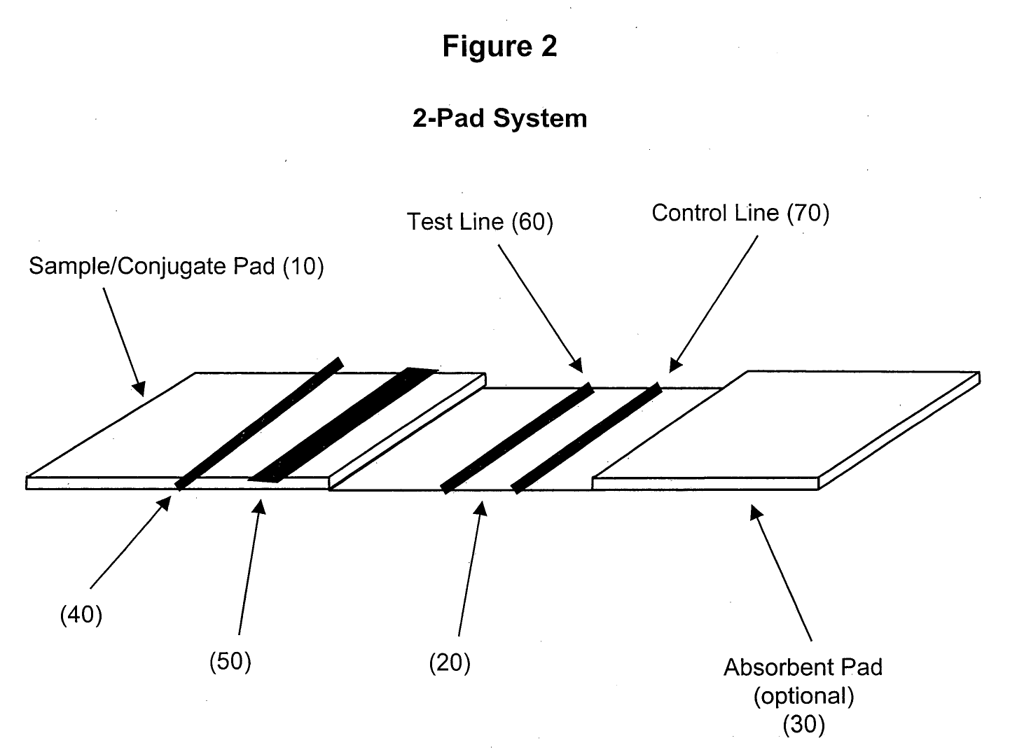

### DETAILED DESCRIPTION

Described herein are devices and assays for detecting an analyte in a liquid sample. In the assay, the target analyte binds an analyte-specific reagent comprising a first member of a conjugate pair, forming a first complex, which then contacts and binds a colored particulate label comprising a complementary member of the conjugate pair, forming a second, particle-labeled second complex. Capture of this analyte-comprising, second complex by an immobilized analyte specific capture reagent results in the formation of an immobilized, labeled sandwich complex that can be detected. Among other advantages of the disclosed device is that assay devices specific for a wide range of analyte can all use the same particle conjugate. The specificity in the assay is contributed by non-particle labeled, analyte-specific reagent that can interact with particle conjugate to provide a detectable complex.

### Analyte/Sample

The analytes that the devices and assays described herein are designed to detect is present in a sample which is in liquid form, or a sample which is in a particulate or solid form that can be converted into a liquid form, i.e. through dissolving in a solvent, such as water. The sample can comprise any material, including any biological or organic material, natural or synthetic. Preferably, the sample comprises a bodily fluid such as blood, urine, saliva, etc.

An analyte encompassed by the assays and devices described herein includes a wide range of molecules that can be dissolved in a fluid compatible with the assays, devices and kits described herein, including, but not limited to compositions and compounds containing a drug, hormone, chemical, toxin, compound, receptor, nucleic acid molecule and fragments thereof, molecules, such as organic and inorganic molecules, peptides, proteins, glycoproteins, an amino acid, carbohydrates, nucleic acids, lipids, toxins, a small molecule, a steroid, a vitamin, an antibody, and combinations thereof. Analytes capable of being detected also include, but are not limited to, neurotransmitters, growth factors, antineoplastic agents, cytokines, monokines, lymphokines, nutrients, enzymes, receptors, antibacterial agents, antiviral agents, and antifungal agents, detectable components of structured elements such as cells, including all animal and plant cells, and microorganisms, such as fungi, viruses, bacteria including but not limited to all gram positive and gram negative bacteria, and protozoa, any antigenic substances, haptens, antibodies, a natural or synthetic chemical substance, a contaminant, a drug including those administered for therapeutic purposes as well as those administered for illicit purposes, and metabolites, and fragments thereof, or combinations thereof. In one embodiment, the analyte is not an antibody or fragment thereof. In another embodiment the analyte will have at least one epitope that an antibody or an immunologically reactive fragment thereof can recognize. Alternatively, or in addition, the analyte contains a site that specifically binds a ligand. The presence of more than one analyte can be detected using the devices and assays described herein. The detection of multiple analytes can be performed simultaneously or sequentially.

### Test Strip

A device for use in an assay for detecting an analyte can be comprised of two or more test strips, each of which can comprise one or more porous components, membranes or filters which provides for capillary flow of a liquid sample. The device has a first pad, also called a conjugate pad, and a detection zone. This test strip is capable of wicking a fluid applied thereto by capillary action within the strip, from an upstream conjugate pad and into a downstream detection zone. The strip can have reagents deposited in zones along the longitudinal length of the membrane. Analyte in the sample contacts the reagents located within the test strip as the sample traverses the length of the strip. Test strip components, e.g. porous supports or membranes such as glass fiber filter and nitrocellulose are available from commercial suppliers or can be customized by laboratory personnel skilled in the art, or by a commercial immunodiagnostic supplier, to include immunoreagents specific for the analyte to be detected. The immunoassay reactions conducted on the strip can include an indirect sandwich immunoassay format.

The test strip of the device comprises a first pad (conjugate pad) and a detection zone which are positioned to permit capillary flow of an aqueous solution from the first pad (conjugate pad) to the detection zone. The first pad (conjugate pad) comprises a porous structure through which the liquid solution is capable of flowing by capillary action from the first pad to the detection zone. The liquid is preferably an aqueous solution in which may be dissolved one or more of the following species at various time points during the assay: one or more analytes of interest, one or more complexes comprising analyte specific reagent(s), and one or more complexes comprising a colored particulate label(s).

The first pad (conjugate pad) of the test strip has a first zone and a second zone, which are separate from each other. The first and second zones are preferably separated by some distances, but can alternatively abut each other. The first zone has a discrete area containing dry, reversibly immobilized reagent capable of specifically binding the analyte. In one instance, the analyte specific reagent is an antibody, or fragment thereof, which is specific for the analyte. In another instance the analyte specific reagent contains a ligand which specifically binds the analyte. The analyte specific reagent also contains a first member of a conjugate pair. In one instance, the first zone contains a single species of an analyte specific reagent located in a discrete area in the first zone.

In another instance, the first zone contains more than one analyte-specific reagent, at least some of which are not identical to each other. In some instances each non-identical analyte-specific reagent specifically binds a distinct and different analyte. Alternatively, or additionally, the first zone contains non-identical, analyte-specific reagents that specifically bind a different epitope or ligand binding area on a single analyte.

In some embodiments, all of the non identical, analyte-specific reagents are located in a single discrete area in zone 1. In other embodiments, each distinct analyte specific reagent is located in a discrete area of its own in zone 1. In other embodiments, zone 1 contains one or more discrete areas that contain identical analyte-specific reagents, and one or more discrete areas which contain non-identical analyte specific reagents.

In one aspect, each non identical, analyte-specific reagent contains the same first conjugate member. In another aspect, only those non identical, analyte-specific reagents which bind different regions on a given analyte contain the same first conjugate member, while the nonidentical, analyte-specific reagents which bind distinct and different analytes, each comprise a different first conjugate member. In another aspect, each non identical, analyte-specific reagent contains a distinct different first conjugate member.

The second zone has a discrete area containing a mobilizable reagent comprising a colored particle label and a complementary member of the conjugate pair.

The second zone of the first pad (conjugate pad) is located downstream from the first zone with respect to the capillary flow of the liquid sample, and has a discrete area containing a mobilizable reagent comprising a complementary member of the conjugate pair and a colored particle label. In a preferred embodiment, the biotin-labeled reagent comes first, with the colored particle second in order from the end to which sample is applied; however, the assay will also work if the particle-labeled reagent is first and the biotin-labeled reagent second.

In one instance, the second zone of the first pad contains a single species of a reagent containing a complementary member of the conjugate pair and a colored particle label, and this single species is located in a discrete area in the second zone, or alternatively in multiple discrete areas in the second zone. In another instance, the second zone of the first pad contains several non identical reagents containing second conjugate members and a colored particle label. In one aspect of this instance, each of the non identical reagents comprise the same conjugate, but different colored particles. In another aspect of this instance, each of the non identical reagents comprises the same colored particle but different conjugate pairs. In yet another aspect, these non identical reagents comprise a mixture of the previous two mentioned aspects.

In further alternative aspects, the indirect labeling can involve, for example, one member of the conjugate pair dried in a discrete zone of the conjugate pad and the other member in liquid suspension prior to running the assay. For example, the particle-labeled member of the conjugate pair can be in liquid suspension, and the other member of the conjugate pair can be dried on the conjugate pad, or alternatively on the second pad, upstream of the detection zone. The order of conjugate pair members can also be reversed, with the particle-labeled member of the conjugate dried on the conjugate pad or the second pad upstream of the detection zone and the other member of the conjugate pair in liquid suspension. In this alternative aspect, the device and the liquid suspension comprising one member of the conjugate pair can be supplied as a kit. For example the kit can comprise a device comprising a porous material which provides for capillary flow of a liquid sample, the device comprising: (1) a first pad (conjugate pad) comprising, in the dry state, a mobilizable reagent comprising one of: a) a colored particle and a first member of a conjugate pair; or b) a reagent capable of specifically binding the analyte and comprising a complementary member of the conjugate pair; and (2) a detection zone comprising a capture line comprising an immobile capture reagent capable of specifically binding analyte. Such a kit would also include a liquid comprising that member of (a) or (b) described above which is not present on the first pad of the device. In use, the liquid including the reagent of (a) or (b) and the liquid sample are contacted with the first pad of the device to run the assay and provide a test result. The liquid including the reagent of (a) or (b) can be mixed with the liquid sample prior to application to the first pad.

The detection zone has a discrete area referred to herein as a capture line, which contains an immobile capture reagent capable of specifically binding analyte. The detection zone preferably also has a discrete area referred to herein as a control line, which contains an immobile capture reagent capable of specifically binding one or more of the mobile analyte-specific reagents of zone 1 of the conjugate pad.

The device provides for capillary flow of the liquid sample from the first pad (conjugate pad) to the detection zone. It is preferable that the flow rate of an aqueous sample through the porous carrier material permits interactions between analyte, analyte-specific reagent and the members of the conjugate pair sufficient to obtain the complexes necessary to generate a detectable signal if analyte is present. The spatial separation between the zones, and the flow rate characteristics of the porous carrier material can be selected to allow adequate reaction times during which the necessary specific binding can occur, and to allow the labeled reagent in the first zone to dissolve or disperse in the liquid sample and migrate through the carrier. Further control over these parameters can be achieved by the incorporation of viscosity modifiers (e.g. sugars, mannitol, modified celluloses, etc.) in the sample or, e.g., on the first pad upstream of the first zone, to slow down the reagent migration.

A device as described herein incorporates two or more discrete sheets of porous material, e.g. separate strips or sheets, one comprising the first pad (conjugate pad) and the second comprising the detection zone. Alternatively, discrete sheets, each sheet comprising a conjugate pad and a detection zone, can be arranged in parallel, for example, such that a single application of liquid sample to the device initiates sample flow in the discrete sheets simultaneously. The separate analytical results that can be determined in this way can be used as control results, or if different reagents are used on the different carriers, the simultaneous determination of a plurality of analytes in a single sample can be made. Alternatively, multiple samples can be applied individually to an array of strips and analyzed simultaneously.

The porous material or porous structure of the device providing the capillary movement or lateral flow includes material such as nitrocellulose, nitrocellulose blends with polyester or cellulose, untreated paper, porous paper, rayon, glass fiber, acrylonitrile copolymer or nylon or other porous materials that allow lateral flow.

In one embodiment, the membrane is a non-woven substrate upon which the reagents can be immobilized or deposited, and which is capable of conveying sample in a fluid flow direction generally parallel to the longitudinal length of the test strip. Desirable test strips are composed of a fluid-conducting material including, but not limited to, nylon, polyethylene, glass fiber, nitrocellulose, cellulose, and other common membrane matrices or bibulous materials. In one embodiment, the test strip is composed of nitrocellulose. In one embodiment, the nitrocellulose sheet has a pore size of at least about 1 micron, or greater than about 5 microns, or about 8-12 microns. In addition, a wide variety of organic and inorganic polymers, both natural and synthetic, may be employed as the material for the solid surface. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, glass fiber filter paper.

It is known in the art that non-bibulous lateral flow can be used to conduct visible moieties, especially labeled particles, e.g., dyed latex. Non-bibulous flow can be achieved using a bibulous support made nonbibulous by treatment with a blocking agent, as is known in the art. Non-bibulous membranes that allow lateral flow of particles are known to one of skill in the art and include, but are not limited to material such as polysulfone microporous membrane, nitrocellulose, cellulose acetate membrane, polyvinyl chloride, polyvinyl acetate, copolymers of vinyl acetate and vinyl chloride, polyamide, polycarbonate, nylon, orlon, polyester, polyester, polystyrene, and the like, or blends can also be used. The selection of material with the desirable properties and flow rate is generally within the knowledge of those skilled in the art.

In one embodiment, the device does not include a housing or casing for the test strip materials. In one embodiment, the test strip materials can be “backed,” or laminated to a semi-rigid support, e.g. a plastic sheet, to increase its handling strength. The material can be one continuous piece or separate pieces. The laminate is preferably polyethylene or vinyl but one skilled in the art will recognize that numerous materials can be used to provide a semi-rigid support. This can be manufactured, e.g., by forming a thin layer of nitrocellulose on a sheet of backing material.

In order to produce adequate mechanical strength or support for the device to function effectively in the collection and analysis of the analyte in the liquid sample, it is necessary that the assay device have an adequate mechanical strength to support the assay device. An adequate mechanical strength is provided by an adequate thickness to the backing material, as well as adequate bending characteristics when using weighted standards or bending characteristics when using water flow measurements. Generally, a minimum adequate mechanical strength is provided by semi-rigid material. Of course, in embodiments in which the device is employed as a dipstick, less rigid backing can be used if desired, as long as it provides a support that is easily handled and stands up to the demands of, e.g., packaging and shipping.

### Analyte-Specific Reagents

Analyte-specific reagents, preferentially analyte-specific antibodies, are reversibly bound to the first zone of the device, while analyte-specific reagents, preferentially analyte-specific antibodies, are irreversibly bound to the capture line of the detection zone of the device. Reversibly bound analyte-specific reagents are retained in zone 1 of the conjugate pad when the material is in the dry state, but is free to migrate through the carrier material when the material is moistened, for example, by the application of liquid sample potentially containing the analyte to be determined.

To permit mobilization of the analyte specific reagent and the particle label when sample is applied to the test strip, several different approaches can be taken, For example, the reagents can be deposited on material that has been pre-treated with a blocking agent to prevent tight non-specific binding of reagent, or the reagents can be deposited in a porous structure with relatively large pores, e.g., a glass fiber filter that permits free transit of particles. The porous structure can be blocked to further prevent binding of assay reagents. Glazing can be achieved, for example, by depositing an aqueous sugar or cellulose solution, e.g. of sucrose or lactose, on the carrier at the relevant portion, and drying. The labeled reagent can then be applied to the glazed portion.

The capturing and control reagents may be striped onto the nitrocellulose or covalently bound or non-covalently attached through nonspecific bonding. Noncovalent binding is typically nonspecific absorption of a compound to the surface. The manner of binding or linking a wide variety of compounds to various surfaces is well known and is amply illustrated in the literature. See, also, for example, IMMOBILIZED ENZYMES, Ichiro Chibata, Halsted Press, New York, 1978, and Cuatrecasas, J. Biol. Chem. 1970 June; 245(12):3059-65, the disclosures of which are incorporated herein by reference.

Reagents can be applied to the membrane materials in a variety of ways that are well known in the art. Various “printing” techniques are suitable for application of liquid reagents to the membranes, e.g. micro-syringes, pens using metered pumps, direct printing, ink-jet printing, air-brush, and contact (or filament) methods, and any of these techniques can be used in the present context. To facilitate manufacture, the membrane can be treated with the reagents and then subdivided into smaller portions (e.g. small narrow strips each embodying the required reagent-containing zones) to provide a plurality of identical carrier units.

### Antibodies

In one embodiment, the reagents of the present invention can be polyclonal and monoclonal antibodies made in vitro or in vivo. Methods of making such antibodies are known in the art.

Polyclonal antibodies against target analytes are generated by immunizing a suitable animal, such as a mouse, rat, rabbit, sheep, goat or chicken, with the analyte of interest. In order to enhance immunogenicity, the peptide can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the peptide may be conjugated to a bacterial toxoid, such as toxoid from diphtheria, tetanus, cholera, etc., in order to enhance the immunogenicity thereof.

### Conjugate Pairs

A first member of a conjugate pair is one member of a conjugate pair, i.e. two molecules, usually two different molecules, where one of the molecules (i.e., a first member of a conjugate pair) through chemical or physical means specifically binds to the other molecule (i.e., a second member of a conjugate pair). The cognate or complementary members of a conjugate pair can include for example, a ligand and its receptor; a receptor and a counter-receptor. The complementary conjugate pairs can include for example: carbohydrates and lectins; complementary nucleotide sequences; peptide ligands and receptor; effector and receptor molecules; hormones and hormone binding proteins; enzyme cofactors and enzymes; enzyme inhibitors and enzymes; etc. The complementary conjugate pairs may include analogs, derivatives and fragments of one or both members of the original conjugate member. The complementary conjugate pairs may also include antibody/antigen interaction where the antibody does not bind an analyte of the assay. In some aspects, however, members of the cognate pair do not include an antibody. For example, a receptor and ligand pair may include peptide fragments, chemically synthesized peptidomimetics, labeled protein, derivatized protein, etc. A preferred conjugate pair for present purposes is biotin/strepavidin. Biotin, avidin or streptavidin (or analogs thereof, e.g., Neutravidin™) can be linked to the colored particles (see below) or to the analyte-specific antibody, by methods known in the art, or using kits such as Pierce (Cat #21338) or Sigma (Cat #B-TAG).

### Colored Particles

The devices and assays described herein preferentially utilize naturally colored or dyed particles as a label, as well as chromogenic and fluorescent dyes as labels. Suggested particles include dyed latex beads, dye imbibed liposomes, metal sols, and the like. The colored particle in such complexes serves as a visible marker, where separation, capture, or aggregation of the particles occurs through binding of a member of a conjugate pair on the particle to an entity bearing a conjugate member of the pair. The amount of label thus segregated in a particular assay step can be related to the amount of analyte initially present in the sample.

Examples of colored particles that can be used include, but are not limited to, organic polymer latex particles, such as polystyrene latex beads, colloidal gold particles; colloidal sulphur particles; colloidal selenium particles; colloidal barium sulfate particles; colloidal iron sulfate particles; metal iodate particles; silver halide particles; silica particles; colloidal metal (hydrous) oxide particles; colloidal metal sulfide particles; carbon black particles, colloidal lead selenide particles; colloidal cadmium selenide particles; colloidal metal phosphate particles; colloidal metal ferrite particles; any of the above-mentioned colloidal particles coated with organic or inorganic layers; protein or peptide molecules or liposomes. Colloidal gold particles may be made by any conventional method, such as the methods outlined in G. Frens, 1973 Nature Physical Science, 241:20 (1973). Alternative methods are described in U.S. Pat. Nos. 5,578,577, 5,141,850; 4,775,636; 4,853,335; 4,859,612; 5,079,172; 5,202,267; 5,514,602; 5,616,467; 5,681,775. Carbon black particles may be attached by methods well known to those skilled in the art, including the methods described, in U.S. Pat. Nos. 5,252,496, 5,559,041, 5,529,901, 5,294,370, 5,348,891 and 5,641,689. Metal sols are described in for example, U.S. Pat. No. 4,313,734. For details and engineering principles involved in the synthesis of colored particle conjugates see Horisberger, Evaluation of Colloidal Gold as a Cytochromic Marker for Transmission and scanning Electron Microscopy, Biol. Cellulaire, 36, 253-258 (1979); Leuvering et al, Sol Particle Immunoassay, J. Immunoassay 1 (1), 77-91 (1980), and Frens, Controlled Nucleation for the Regulation of the Particle Size in Monodisperse Gold Suspensions, Nature, Physical Science, 241, pp. 20-22 (1973).

A cognate pair as described herein comprises a first member and a second member. One member is conjugated to a particulate label. In one aspect, the member that is conjugated to a particulate label is not an antibody specific for the first member of the pair. In another aspect of this embodiment, the particles are not labeled with an antibody that specifically binds biotin. Non-antibody-labeled particles may confer benefits including, e.g., reduced background.

Colored latex particles can be produced either by incorporating a suitable dye, such as anthraquinone, in the emulsion before polymerization, or by coloring the pre-formed particles. In the latter route, the dye should be dissolved in a water-immiscible solvent, such a chloroform, which is then added to an aqueous suspension of the latex particles. The particles take up the non-aqueous solvent and the dye, and can then be dried.

Preparation of the latex particle conjugates can be accomplished, e.g., by immobilizing the conjugate such as avidin to 0.43 μm microspheres (Magsphere carboxylated PS) to produce conjugate pair member-latex conjugates. For example, to a 1% suspension of 0.43 μm carboxylated microspheres, one would add 0.15 mg/mL of N-(3-Dimethylaminopropyl)-N′-ethylcarbodiimide hydrochloride (EDAC, Sigma E1769) and 1:40 dilution (wt/wt) of the conjugate pair, e.g., avidin, streptavidin, or neutravidin in a 50 mM Borate buffer, pH 8.5. This reaction is allowed to incubate while stirring or rotating for 3 hours at room temperature. Excess EDAC and avidin are removed by centrifugation at 12,000 RPM for 15 minutes, followed by re-suspension in a protein containing storage solution (20 mM Tris, 0.1% NaN₃, 2% Casein, 10% Sucrose). The latex beads can be applied on the material by using airjet techniques such as a BioDot Biodoser machine from Bio-Dot, Inc., Irvine, Calif. Such application can allow the labeling reagents to be mobile.

The selection of particle size may be influenced by such factors as stability of bulk sol reagent and its conjugates, efficiency and completeness of release of particles from (conjugate pad), speed and completeness of the reaction. Also, the fact that particle surface area may influence steric hindrance, between bound moieties may be considered. Particle size may also be selected based on the porosity of the porous material of the test strip and/or application pad. The particles are preferably sufficiently small to diffuse along or through the membrane or support by capillary action of a sample liquid. Preferably, the direct label is a colored latex particle of spherical or near-spherical shape and having a maximum diameter of not greater than about 0.5 micron. Preferably, the size range for such particles is from about 0.05 to about 0.5 microns. Coupling of the particulate label to a conjugate member to form a colored particulate label conjugate can be by covalent bonding or by hydrophobic bonding.

Latex (polymer) particles for use in immunoassays are available commercially. These can be based on a range of synthetic polymers, such as polystyrene, polyvinyltoluene, polystyrene-acrylic acid and polyacrolein. The monomers used are normally water-insoluble, and are emulsified in aqueous surfactant so that monomer mycelles are formed, which are then induced to polymerize by the addition of initiator to the emulsion. Substantially spherical polymer particles are produced.

The number of labeled particles present in the porous material of either the first pad (conjugate pad) and/or the analyte detection membrane support or test strip may vary, depending upon the size and composition of the particles, the composition of the test strip and porous material of either the first pad (conjugate pad) or the analyte detection membrane or support, and the level of sensitivity of the assay. Particles can be labeled in addition to being colored, to facilitate detection. Examples of labels include, but are not limited to, luminescent labels; colorimetric labels, such as dyes; fluorescent labels; or chemical labels, such as electroactive agents (e.g., ferrocyanide). Colored particle labels such as colored polystyrene particles can be deposited in zone two of the conjugate pad by a similar process to that described above for reversibly binding an analyte-specific reagent of zone 1 of the conjugate pad.

An advantage of the disclosed indirect labeling approach is that the same particulate-labeled conjugate can be used as a sort of “universal reagent” for the preparation of assay devices to detect multiple different analytes. That is, multiple different analyte-specific reagents can be separately conjugated to the same member of a conjugate pair (e.g., biotin). Each of these separate analyte-specific reagents will be indirectly labeled by the same particle-labeled conjugate having the cognate member of the conjugate pair (in this instance, streptavidin). This eliminates the need to prepare multiple different (analyte-specific) particulate-labeled preparations in order to detect multiple different analytes.

### Detection Zone

The detection zone is located downstream of the first pad (conjugate pad) with respect to the capillary movement of the liquid sample. The detection zone has at least one capture line which contains an immobilized capture reagent irreversibly bound to a discrete area. The immobilized capture reagent is capable of specifically binding to the analyte. The capture reagent can be an antibody or fragment thereof, which is capable of specifically binding to the analyte. The capture reagent can also be a ligand, natural or synthetic, which is capable of specifically binding to the analyte. A single capture line in the detection zone any contain nonidentical immobilized capture reagents, for example capture reagents which recognize distinct parts of the analyte. The detection zone can contain more than one capture line. In instances where the detection zone contains more than one capture line, each capture line can contain a distinct, nonidentical, irreversibly bound capture reagent which specifically binds a distinct analyte. Alternatively, in instances where the detection zone contains more than one capture line, each capture line can contain a distinct, nonidentical, irreversibly bound, capture reagent which specifically binds a different epitope or region of the analyte. Alternately, in instances where the detection zone contains more than one capture line, each capture line can contain a mixture of nonidentical capture reagents.

The detection zone of the device optionally further comprises a control line which is preferably located downstream of the capture line with respect to the capillary movement of the liquid sample. The control line is a discrete area of the detection zone and contains a reagent which is irreversibly immobilized to the control line and which specifically binds to the reagent of the first zone of the first pad (conjugate pad). In one aspect, the analyte-specific reagent of the first zone of the first pad (conjugate pad) is an antibody, and the control line comprises a reagent which specifically binds antibodies. In another aspect, the reagent comprises a final capture antibody, wherein said final capture antibody specifically binds to antibody molecules depending on their specificity. In another aspect, the final capture antibody binds antibody regardless of its specificity. In aspects where there are multiple analyte-specific reagents, each specific for one of a multiplicity of different analytes of interest, there can be multiple control lines, each line containing one or more reagents specific for at least one of the multiple analyte specific reagents.

It may be desirable to assay two or more different analytes using the same device. In such instances, it may be desirable to employ different detectable markers on the same test strip where each detectable marker detects a different analyte. For example, different analyte-specific reagents may be attached to different colored particle labels so as to form distinguishable complexes. For example, the complexes can contain different detectable markers, e.g., different fluorescent agents which fluoresce at different wavelengths, or differently colored dyes or particles. When detecting two or more different analytes using the same device, separate capture lines and/or control lines can optionally be formed in the detection zone on the test strip for each analyte to be detected. Alternatively, the same detectable marker can be used for all of the analytes. Alternatively, different detectable markers, as described above, can be used for the different analytes to prevent one capture zone being confused with another.

The accumulation of visible labels can be assessed either visually or by optical detection devices. Retention of label in the capture line indicates the presence of target analyte in the sample. Retention of label in the control zone indicates that sufficient fluid has passed through the first pad (conjugate pad) and detection zone, and that the labeled reagent is not denatured or degraded due to storage, buffer composition and sample composition, etc.

In addition, the visible intensity of the accumulated labels can be correlated with the concentration or titer (dilution) of analyte in the patient sample. The correlation between the visible intensity of accumulated labels and analyte concentration can be made by comparison of the visible intensity to a reference standard. Thus, analyte levels can be determined by devices as described herein.

The device described herein can optionally further comprise a sample pad, which has a porous structure allowing a liquid sample comprising one or more analytes of interest to flow by capillary action. A sample pad is not generally necessary. However, where used, the sample pad is positioned to allow the liquid sample to flow to the first pad (conjugate pad). The sample pad can partially or fully overlap the first pad (conjugate pad).

A reagent sink or absorbent pad is optionally included at the distal end of the test strip for enhancing the flow of the fluids, including reagents and sample, along the longitudinal length of the strip. The absorbent zone or reagent sink is a means for removing excess fluid from the matrix of the device, thus maintaining the desired capillary flow along the flow path. The sink can be composed of an absorbent material, such as blotting paper, filter paper, a glass fiber filter, or the like.

### Methods

In general, a method for determining the presence or absence of an analyte in a sample, using a lateral flow device described herein, comprises:

(a) if necessary, treating the sample so as to extract the analyte into an aqueous solution;

(b) applying the aqueous sample to the device;

(c) waiting a predetermined amount of time to allow the sample to reach the detection zone in the test strip; and

(d) viewing the results on the test strip, the results indicating the presence or absence of the analyte in the sample.

In one preferred assay format, the contacting step includes placing the sample on a first pad of a dry strip having, in an upstream to downstream direction, a first zone containing reversibly bound labeled antibody which specifically binds the analyte of interest, and which comprises a first member of a conjugate pair, and a second zone comprising a dry, reversibly bound colored particulate label, wherein the particulate label comprises a second member of the conjugate pair, wherein the second member is complementary to the first member of the conjugate pair, and a detection zone containing immobilized irreversibly bound antibody specific for the analyte in the complex formed by the conjugated, analyte-specific antibody with the conjugated particulate label.

In operation, (i) sample migrates in a downstream direction on the conjugate pad toward the first zone, (ii) the analyte in the aqueous solution specifically reacts with the reversibly bound, analyte-specific, conjugated antibody, forming a complex which migrates toward the second zone wherein the complex specifically binds a colored particulate label comprising the complementary member of the conjugate pair, forming a complex wherein the analyte is indirectly bound to a particulate colored label through a pair of conjugated members. This latter complex migrates to the detection zone where the complex contacts and specifically binds to the analyte specific antibody irreversibly bound in the capture line, thus capturing the complex comprising the analyte and a detectable colored particle at the capture line. Complexes without analyte comprising analyte-specific antibody bound to a particulate colored label through a pair of conjugated members, are not captured and migrates downstream of the capture zone to the control line, the control line comprising antibody which is irreversibly bound to the control line and which specifically binds antibody molecules regardless of the idiotype of the antibody. The detecting step in both the capture and control lines includes detecting the presence or absence of immobilized, labeled complex in each zone, the presence of detectable label in the capture line of the detection zone indicating the presence of the analyte in the aqueous solution applied to the device.

### Kits

A kit for the detection of analyte in a sample provided herein contains a device described herein and optionally, a fluid, such as a buffer to be combined with sample suspected of containing the analyte to form an aqueous solution or liquid suspension. The kit may additionally contain additional reagents or buffers, equipment for obtaining or collecting the sample, a vessel for containing the sample and reagents, a timing means, and/or a standard against which a color change may be measured. The presence or absence of the analyte in the aqueous solution is determined by the presence or absence of detectable label in the capture line of the detection zone.

### EXAMPLE

### Preparation of One-Step Assay for Group A Streptococcus

### Selection of Materials

1. Analyte Detection Region: Important features of the material are its fluid wicking and protein binding abilities. Exemplary material includes nitrocellulose, nylon or the like. In a preferred embodiment, the material is nitrocellulose with or without laminated solid support such as polyester. Nitrocellulose is readily available from numerous suppliers.

2. Conjugate pad: Suitable materials include cotton, cellulose, mixed fibers, glass fiber and the like. For example, paper such as 470 and 740-E from Schleicher and Schuell, Keene, N. H., or D28 from Whatman, Fairfield, N.J., can be selected for its high fluid absorption and wicking speed. A more porous material such as glass fiber #66078 from Gelman Sciences, Ann Arbor, Mich., Grade 9818 glass fiber from Lydall Inc., Manchester, Conn., or POREX from Porex Technologies, Fairbum, Ga., is suitable for impregnating with the conjugated colored labeled particles of Zone 2 of the conjugate pad and for impregnating with the conjugated analyte specific reagent of zone 1 of the conjugate pad.

3. Backing Supports: For the devices described herein, the preferred materials are clear mylar with thickness about 0.001 inches to 0.010 inches for an optional upper covering and white vinyl with thickness about 0.005 inches to 0.030 inches for the lower backing. Both the mylar and the vinyl sheets have adhesive on one side so as to attach the porous material. Materials such as mylar, polyester, and vinyl with adhesive are readily available.

4. Reagents:

A) A chromogenic particulate, such as latex, is labeled with a cognate member (avidin) of a conjugate pair, suitable for reacting with a first member of a conjugate pair of the analyte specific reagent located in zone 1 of the conjugate pad.

B) Antibody directed to Strep A can be obtained from commercial sources such as Fitzgerald Industries (Concord, Mass.), Lampire Biological Lab (Pipersville, Pa.), or preferably polyclonal antibodies can be raised in-house. Affinity purified and pepsin digested antibody directed to Strep A is conjugated to biotin.

C) In-house generated, affinity purified anti-Strep A antibody is irreversibly bound to the capture line in the detection zone.

D) Polyclonal antibody specific to an unrelated protein (e.g. BSA, GSA, ovalbumin, mouse immunoglobulin) is irreversibly bound to the control line.

### Preparation of Latex Conjugates

The basic protocol for conjugation of protein to latex, by simple adsorption or by covalent binding, is well known in the art and is hereby incorporated by reference. As an example, covalent attachment of avidin to squaraine-dyed latex beads is described in, e.g., U.S. Pat. Nos. 5,536,644, 5,076,950, 4,935,147, and 5,370,993.

Preparation of the latex particle conjugates can be accomplished, e.g., by immobilizing the conjugate such as avidin to 0.43 μm microspheres (Magsphere carboxylated PS) to produce conjugate pair member-latex conjugates. For example, to a 1% suspension of 0.43 μm carboxylated microspheres, one would add 0.15 mg/mL of N-(3-Dimethylaminopropyl)-N′-ethylcarbodiimide hydrochloride (EDAC, Sigma E1769) and 1:40 dilution (wt/wt) of the conjugate pair member, e.g., avidin, streptavidin, or neutravidin in a 50 mM Borate buffer, pH 8.5. This reaction is allowed to incubate while stirring or rotating for 3 hours at room temperature. Excess EDAC and avidin are removed by centrifugation at 12,000 RPM for 15 minutes, followed by re-suspension in a protein containing storage solution (20 mM Tris, 0.1% NaN₃, 2% Casein, 10% Sucrose). The latex beads can be applied on the material by using airjet techniques such as a BioDot Biodoser machine from Bio-Dot, Inc., Irvine, Calif. Such application can allow the labeling reagents to be mobile.

### Application of Affinity Purified Polyclonal Anti-Strep a Antibody (Capture Reagent) on the Capture Line of the Detection Zone, and of Anti-BSA Antibody (Final Capture Reagent) on the Control Line of the Detection Zone:

Thin lines of the antibodies are applied to the capture line or control line, respectively, on the material using pipetting techniques, which include application with flat-tipped pipet tips. Alternative methods can be application by airbrush techniques (Iwata, model HP-BC2). The width of the lines can be, e.g., 0.2 mm to 2 mm; a width of 1 mm is preferred. Such material is immobilized by techniques well known in the art.

### Application of Latex-Avidin Conjugated Particles, BSA Coated Particles, and the Biotin Conjugated Anti Strep A Antibody to Zone 2 and Zone 1, Respectively, of the First Pad (Conjugate Pad)

Avidin and BSA conjugated latex particles, and biotin labeled anti-Strep A antibodies are applied to Zone 2 and Zone 1, respectively, of the first pad (conjugate pad). The latex solution can be applied on the material by pipetting or airbrushing techniques. The membrane strip is then dried, e.g., by forced air or by lyophilization. Such application allows the labeling reagents to be mobile.

Stabilizing agents can also be included in the application solution to protect reagents, e.g., antibody during drying or lyophilization. Stabilizers can include, e.g., sugars, e.g., sucrose, lactose, etc., proteins, e.g., BSA, casein, etc., gelatin, PVA, amino acids, or detergents, e.g., TWEEN and mannitol.

### Further Preparation of the First Pad (Conjugate Pad)

The first pad (conjugate pad) can further be treated with buffer containing detergents, blocking proteins and the like to facilitate movement of dried latex particles, neutralize extraction reagents or to reduce nonspecific binding of the assay. In the case of the Strep A assay, an appropriate amount of buffer solution is dispensed to the first pad, dried, and then assembled into the assay device. For example, 1.6M Tris with 0.1 M NaCl, 0.1% Sodium Azide, 1.5% Zwittergent, and 0.1% Rabbit IgG can be applied to the first pad. The first pad (conjugate pad) is then dried in a forced air oven.

### Assembly of the Assay Device

A sheet of white vinyl (98 mm×254 mm) is placed on a flat surface. The cover paper on the white vinyl sheet is removed to expose the adhesive. A strip of material (25 mm×254 mm), the first pad (conjugate pad) containing avidin and BSA conjugated latex and anti-Strep A antibody lines is attached to the white vinyl sheet. The analyte detection region is positioned downstream of the first pad (conjugate pad) with respect to capillary flow of the liquid sample. The absorbent pad is attached to the right edge of the white vinyl sheet (downstream of the capillary flow) while overlapping about 3 mm on top of the analyte detection region (downstream of capture and control lines). The cover paper from the clear mylar sheet is removed (98 mm×254 mm) to expose the adhesive. Lining up to the right edge, the cover is attached to the clear mylar sheet with the adhesive side down on top of the end flow region, analyte detection region and sample receiving region. The whole sheet is pressed with a roller to ensure the lamination is secure. The laminated sheet is then cut to 3.8 mm wide sticks.

### Procedure for Testing the Presence or Absence of Strep A from Extracted Samples

Swab samples must have the Strep A antigen extracted into a solution-filled container, into which the device can be dipped (dipstick format). Within five minutes, the test result if positive for Strep A will appear as one blue line on the capture line and one red line on the control line. If only a red line appears on the control line, then the results are negative. The control line is used as a control to ensure the assay reagents are working and that lateral flow is occurring.

One skilled in the art will readily appreciate that the present invention is well adapted to carry out the objects and obtain the ends and advantages mentioned as well as those inherent therein. The immunological methods and devices for detecting analytes in biological samples as described herein are presently representative of preferred embodiments, are exemplary and not intended as limitations on the scope of the invention. Changes therein and other uses will occur to those skilled in the art which are encompassed within the spirit of the invention or defined by this scope with the claims.

It will be readily apparent to one skilled in the art that varying substitutions and modifications may be made to the invention disclosed herein without departing from the scope and spirit of the invention. All references and citations disclosed herein are incorporated by reference in their entirety.

1. A device for detecting an analyte in a liquid sample, wherein said device comprises: porous material which provides for capillary flow of a liquid sample, said device comprising:
(A) a first pad (conjugate pad) comprising a first and second zone, one of said first zone or second zone comprising in the dry state, a mobilizable reagent capable of specifically binding said analyte, wherein said reagent further comprises a first member of a conjugate pair, the other of said first zone or said second zone comprising a mobilizable reagent comprising a colored particle label and a complementary member of said conjugate pair;
(B) a detection zone comprising a capture line comprising an immobile capture reagent capable of specifically binding analyte,
wherein said device provides for capillary flow of the liquid sample from the first pad (conjugate pad) to the detection zone.

2. The device of claim 1, wherein said mobilizable reagent of said first zone comprises an antibody which specifically binds analyte.

3. The method of claim 2, wherein said immobile capture reagent comprises an antibody which specifically binds analyte.

4. The device of claim 1, wherein said detection zone further comprises a control line comprising a final capture reagent capable of binding the mobilizable reagent of said second zone.

5. The method of claim 4, wherein said final capture reagent is capable of specifically binding an antibody, regardless of the antibody's antigen specificity.

6. The device of claim 5, wherein said first member of said first conjugate pair is biotin, and wherein said complementary member of said conjugate pair is selected from the group consisting of streptavidin, neutravidin and avidin.

7. The device of claim 1, further comprising a material capable of absorbing excess liquid, said material being located in a position in the device to provide for capillary flow of said sample from its application point through the detection zone.

8. An assay device for detecting an immunoreactive analyte present in an aqueous solution, said device comprising a first pad (conjugate pad) and a detection zone, wherein said first pad (conjugate pad) and said detection zone are positioned to permit capillary flow of an aqueous solution from said first pad (conjugate pad) to said detection zone,
(1) wherein said first pad (conjugate pad) comprises a porous structure through which an aqueous solution is capable of flowing by capillary action, wherein said first pad (conjugate pad) comprises a first and a second zone, said first zone being separate from said second zone, wherein, in the dried, unused state,
(a) at least one of said first zone or said second zone comprises a dry, reversibly immobilized antibody specific for said analyte, said antibody comprising a first member of a conjugate pair,
(b) and the other of said first zone or said second zone comprises a dry, reversibly immobilized colored particulate label, said colored particulate label comprising a complementary member of said conjugate pair, and
(2) wherein said detection zone comprises a capture line comprising an irreversibly immobilized capture antibody capable of specifically binding to said immunoreactive analyte.

9. The device of claim 8, which has the functional characteristics as follows:
(i) upon addition of an aqueous sample comprising said analyte to said first pad (conjugate pad) of said device, reversibly immobilized antibody specific for said analyte is mobilized, and a first complex is formed comprising said antibody and said analyte, wherein said first complex, together with unbound antibody, is capable of subsequently moving by capillary action to said second zone,
(ii) wherein upon contact with advancing aqueous sample, said reversibly immobilized colored particulate label is mobilized, permitting binding of said first complex to said particulate label in said second zone to form a second complex comprising said first complex and said particulate label, wherein said second complex is capable of subsequently moving by capillary action to the capture line of said detection zone, and/or
(iii) wherein upon binding of said unbound antibody of part (i) to said particulate label in said second zone, a third complex is formed, said third complex comprising said unbound antibody of part (i) and said particulate label, wherein said third complex is capable of subsequently moving by capillary action to a final capture line of said detection area,
wherein upon said capture antibody's specific binding in aqueous solution to the analyte component of said second complex formed in part (ii), a fourth complex is formed at said final capture line, said fourth complex comprising said second complex and said capture antibody, wherein the formation of said fourth complex is indicative of the presence of said analyte of interest in said aqueous sample.

10. The device of claim 8, wherein said detection area further comprises a control line comprising a reagent which is irreversibly immobilized to the control line and which specifically binds to antibody molecules.

11. The device of claim 8, wherein said control line is located downstream of said capture line with respect to the capillary flow of said aqueous solution.

12. The device of claim 8, wherein said first zone of said first pad (conjugate pad) is positioned upstream of said second zone with respect to the capillary flow of said aqueous solution.

13. The device of claim 8, wherein said first member of said first conjugate pair is biotin, and said second member of said conjugate pair is selected from the group consisting of streptavidin, neutravidin, avidin and anti-biotin.

14. The device of claim 8, wherein said colored particle is selected from the group consisting of latex particles, colloidal gold particles and carbon sol particles.

15. The device of claim 10, wherein said reagent comprises a final capture antibody, wherein said final capture antibody specifically binds to antibody molecules.

16. The device of claim 15, wherein said final capture antibody binds antibody regardless of its antigen specificity.

17. A method of detecting an analyte in an aqueous solution comprising:
A) contacting said solution with an analyte-specific antibody reversibly immobilized to a porous structure under conditions that permit the mobilization of said analyte-specific antibody and the formation of a first complex in which the analyte is specifically bound to the analyte-specific antibody, wherein the analyte-specific antibody comprises a first member of a conjugate pair,
B) wherein said solution, carrying said first complex, permits contacting and mobilization of a colored particulate label reversibly immobilized to said porous structure and located distal to the site of reversible immobilization of said analyte-specific antibody, said colored particle comprising a complementary member of said conjugate pair, under conditions that permit the formation of a second complex in which the first complex is specifically bound to the colored particulate label,
C) wherein upon formation of said second complex, said second complex migrates by capillary action and contacts an analyte specific capture antibody which is irreversibly immobilized to said porous structure at a position distal to the site of formation of said second complex, under conditions that permit the formation of a third complex comprising said second complex and said capture antibody,
D) detecting the formation of said third complex by detecting its particulate label component thereby accumulated on the porous structure,
wherein detection of said third complex indicates the presence of said analyte in said aqueous solution.

18. The method of claim 17, wherein step (B) also comprises contacting analyte-specific antibody of part (A), which has not bound analyte, with the colored particulate label of part (B), under conditions that allow the formation of a fourth complex in which the analyte-specific antibody of part A), which has not bound analyte, is specifically bound to the colored particulate label,
E) wherein upon formation of said fourth complex, contacting said fourth complex with a reagent which specifically binds to antibody molecules which is irreversibly immobilized to said porous structure at a position distal to the site of formation of said second and third complex, under conditions that allow the formation of a fifth complex comprising said fourth complex and said reagent which specifically binds to antibody molecules,
F) detecting the formation of said fifth complex by detecting its particulate label component thereby accumulated on the porous structure,
wherein detection of said fifth complex acts as a control for the functionality of the assay.

19. A method for determining the presence of an analyte of interest in a sample, comprising the following steps:
(A) providing the device of claim 8,
(B) applying an aqueous solution comprising said sample to said device such that said solution contacts the first zone of the first pad (conjugate pad) of said device, thereby providing for, in sequence:
(1) aqueous reconstitution and mobilization of the antibody reversibly bound to said first zone, said antibody comprising a first member of a conjugate pair, wherein said antibody is capable of specifically binding said analyte, and
(2) formation of a first complex upon said analyte's binding specifically to said antibody, said first complex comprising said antibody and said analyte,
(3) movement by capillary action of both said first complex and antibody reconstituted in step (1) which has not specifically bound analyte, to the second zone of said first pad (conjugate pad) of said device,
(4) aqueous reconstitution of the colored particle label reversibly bound to said second zone, said colored particle label comprising a second member complementary to said first member of said conjugate pair,
(5) formation of a second and/or a third complex, wherein:
a) said second complex is formed upon binding of said particulate label to said first complex through said first and second members of said conjugate pair, said second complex comprising said first complex and said particulate label,
b) said third complex is formed upon binding of said particulate label to said unbound antibody of step (3) through said first and second members of said conjugate pair, said third complex comprising said unbound antibody of step (3) and said particulate label,
(6) movement by capillary action of said second and/or said third complex formed in step (5) to the capture line of said detection area of said device,
(7) aqueous reconstitution of the capture antibody irreversibly bound to said capture line, wherein said capture antibody is capable of specifically binding to said analyte,
(8) formation of a fourth complex upon said capture antibody's specific binding to said second complex formed in step 5(a), said fourth complex comprising said second complex and said capture antibody irreversibly bound to said capture line in said detection area,
wherein the formation of said fourth complex is indicative of the presence of said analyte of interest in said sample.

20. The method of claim 19, wherein the third complex formed in step (5)(b) moves by capillary action to a control line which is downstream of said capture line of said detection zone of said device and comprises a final capture reagent, further comprising the steps of:
(9) the subsequent reconstitution of the final capture reagent irreversibly bound to said control line, wherein said capture reagent is capable of specifically binding to an antibody of any specificity,
(10) the subsequent formation of a fifth complex comprising the complex formed in step (5)(b) and the control antibody reconstituted in step (9),
wherein formation of said fifth complex indicates the successful reconstitution of dried reagent reversibly bound to said first zone of said sample pad and of the dried reagent reversibly bound to the second zone of said sample pad, and the formation of a complex comprising the two reagents through their respective members through the pair said device, the successful pairing of and migration of both the antibody in said first zone of said sample the presence of antibody which has not specifically bound the analyte of interest.

21. A kit for detecting an analyte in a liquid sample, wherein said kit comprises:
A) a device comprising a porous material which provides for capillary flow of a liquid sample, said device comprising:
(1) a first pad (conjugate pad) comprising, in the dry state, a mobilizable reagent comprising one of:
a) a colored particle and a first member of a conjugate pair; or
b) a reagent capable of specifically binding said analyte, said reagent capable of specifically binding said analyte further comprising a complementary member of said conjugate pair; and
(2) a detection zone comprising a capture line comprising an immobile capture reagent capable of specifically binding analyte,
wherein said device provides for capillary flow of the liquid sample from the first pad (conjugate pad) to the detection zone; and
B) a liquid comprising that member of (a) or (b) recited above which is not present on said first pad of said device; wherein, in use, said liquid and said liquid sample are contacted with said first pad of said device.

22. The kit of claim 21 wherein said mobilizable reagent comprised by said first pad comprises a colored particle and a first member of said conjugate pair.

23. The kit of claim 21 wherein said mobilizable reagent comprised by said first pad comprises a reagent capable of specifically binding said analyte, said reagent capable of specifically binding said analyte further comprising a complementary member of said conjugate pair.

24. The kit of claim 21 wherein said mobilizable reagent comprises a colored particle and said first member of said conjugate pair, and wherein said liquid of (B) comprises a reagent capable of specifically binding said analyte, said reagent capable of specifically binding said analyte further comprising said complementary member of said conjugate pair.

25. The kit of claim 21 wherein said mobilizable reagent comprises a reagent capable of specifically binding said analyte, said reagent capable of specifically binding said analyte further comprising said complementary member of said conjugate pair, and wherein said liquid of (B) comprises a said colored particle and said first member of said conjugate pair.
